(19) United States
(12) Patent Application Publication
(10) Pub. No.: US 2024/0198091 A1
(43) Pub. Date: Jun. 20, 2024

(54) HIGHLY MICROPOROUS GRAPHENE-BASED NEURAL ELECTRODE

(71) Applicant: GEORGE MASON UNIVERSITY, Fairfax, VA (US)

(72) Inventors: Pilgyu Kang, Fairfax, VA (US); Shirin Movaghgharnezhad, Fairfax, VA (US)

(21) Appl. No.: 18/532,214

(22) Filed: Dec. 7, 2023

Related U.S. Application Data
(60) Provisional application No. 63/430,812, filed on Dec. 7, 2022.

(51) Int. Cl. A61N 1/05; A61N 1/36; C01B 32/184

(52) U.S. Cl. CPC A61N 1/0551; A61N 1/36125; C01B 32/184; C01B 2204/22; C01B 2204/32; C01P 2004/03; C01P 2006/12; C01P 2006/14

(57) ABSTRACT

Highly microporous laser fabricated three-dimensional graphene which can be prepared from a fluorinated polyimide is disclosed as a material suitable for a neural electrode, e.g., a neural stimulation device. The three-dimensional porous graphene incorporated into the device may have a multi-scale structure, which enables electrical and charge carrying properties. In some aspects, the porous graphene incorporated into the device has the following pore structure: macropores having an average pore size exceeding 50 nm; mesopores having an average pore size of 2-50 nm; micropores having an average pore size of 2 nm or less; and nanopores having an average pore size of less than 100 nm. Neural stimulation devices and methods of using the devices are also described.

### CROSS-REFERENCE TO RELATED APPLICATIONS

This application claims priority to U.S. Provisional Application No. 63/430,812, filed Dec. 7, 2022, which is incorporated into this application by reference.

### BACKGROUND

The development of effective therapeutic approaches for treating chronic pain, neural disorders, and addressing post-injury recovery is important. Neural electrodes have emerged as a promising technique for targeted modulation of neural activity and improved patient outcomes. These electrodes offer the potential to selectively activate or inhibit specific neural pathways and are being actively investigated as a treatment strategy for a range of neurological and psychiatric conditions. High-performance neural electrodes are useful for efficient neural stimulation, implying the importance of neural electrodes with high charge storage capacity (CSC) and charge injection capacity (CIC).

While a higher CSC often correlates with an increased CIC due to the greater charge accommodation on the electrode surface and subsequent delivery to tissue, the CIC is also influenced by electrode material, structure, and electrochemical properties at the electrode-tissue interface. Conventional metal-based neural electrodes have limited CIC, ranging from 0.05-0.26 mC cm⁻², due to their relatively small accessible surface area and low double-layer capacitance. This capacitance, a key property dictating charge storage, is constrained by the modest surface area inherent to metal-based electrode designs. Metal-based electrodes exhibit a relatively small accessible surface area, leading to constraints in their double-layer capacitance and, consequently, their CSC.

Among metal choices, platinum (Pt) is the predominant option for neural electrodes, yet it possesses a limited CSC below 1.2 mC cm⁻² and a restricted CIC, typically below 0.15 mC cm⁻², which restricts its efficacy in neural stimulation. As a result, achieving the desired charge delivery to the neural tissue may demand applying higher voltages, thus increasing the potential for electrochemical reactions. These reactions can have detrimental effects on neural tissue, including oxidative stress, inflammation, cell and structural damage, as well as tissue damage.

Various approaches have been employed to enhance the CSC and CIC of Pt electrodes, including the construction of composite materials and the engineering of metallic structures. The synthesis of composite materials, such as Pt—TiN, Pt—IrOx, Pt-PEDOT, and Pt-PEDOT:CNT, has demonstrated enhancement in both CSC and CIC compared to Pt. Composite materials can offer a larger surface area for charge storage because they combine different components, often incorporating nanoscale structures. These structures collectively provide more active sites for charge storage reactions, resulting in an increased surface area and a higher CSC.

Furthermore, composite materials increase conductivity by incorporating conductive materials into the composite, creating pathways for electron flow, and improving overall electrical conductivity. Higher conductivity enhances CIC by facilitating more efficient charge transport, enabling rapid charge injection and transfer at the electrode-electrolyte interface. CSC and CIC have also been enhanced by the porous structuring of metals, such as nanoporous Pt, nanofibrous Pt, called Pt-grass, nanoporous metal oxides, and nanoporous metal nitrides, due to the significant increase in electrochemically active surface area. The increased surface area allows for the accumulation of more electrical charge at the electrode interface, enabling larger charger injection during neural stimulation. However, metal-based electrodes are limited in flexibility and biocompatibility, increasing the potential of tissue damage. Therefore, the use of flexible and biocompatible materials is desired for neural electrodes.

Carbon-based materials, such as carbon nanotubes, carbon fibers, glassy carbons, and graphene exhibit several advantages over metal-based neural electrodes for neural stimulation. They offer enhanced biocompatibility, flexibility, and lower electrical impedance, resulting in efficient charge transport with reduced tissue damage. Among the carbon-based materials, graphene, with high electron mobility and electrical conductivity, mechanical strength, high flexibility, and high transmittance, has been explored as a neural electrode functional material for simultaneous neural stimulation, recording, and imaging applications. However, the interfacial capacitance of non-structured graphene, which measures an electrode's efficiency in storing and releasing electrical charge at the interface between the graphene and the electrolyte, is remarkably low, below 0.02 mC cm⁻², much lower than that of conventional metal electrodes. Unstructured graphene exhibits low CSC and CIC (<1 mC cm⁻²), hindering efficient neural stimulation. A need in the art exists for improvements to graphene-based materials for applications such as neural electrodes.

### SUMMARY

In one aspect, micro-scale porous graphene-based neural electrodes were fabricated using a scalable and fast approach of laser technology from a polyimide film. The electrode showed an enhanced cathodic charge storage (CSC) of 50 mC cm⁻² and charge injection capacity (CIC) of 2 mC cm⁻². Without being bound by theory, it is believed that the improved CSC and CIC is attributable to the electrochemically enhanced active surface area and interconnected porous structure of laser-induced graphene, enabling larger charge storage and more efficient charge transfer compared to monolayer graphene-based neural electrode, which was not prepared from the fluorinated polyimide film.

In one aspect, the disclosed neural stimulation device, comprises: (a) a substrate; (b) a transparent polymer film deposited on the substrate; and (c) at least one layer of porous graphene on or within the transparent polymer film. In some aspects, the at least one layer of porous graphene has the following pore structure: (i) macropores having an average pore size exceeding 50 nm; (ii) mesopores having an average pore size of 2-50 nm; (iii) micropores having an average pore size of 2 nm or less; and (iv) nanopores having an average pore size of less than 100 nm. In a further aspect, the nanopores have a BET specific surface area of at least 300 m²/g.

Also described is a method of stimulating a nerve of a subject, comprising placing the disclosed neural stimulation device within sufficient proximity of the nerve to thereby stimulation the nerve. In some aspects, sufficient proximity can include contacting the nerve with the neural stimulation device.

Also described is a method for making the neural stimulation device, comprising: depositing a transparent polymer film onto a substrate, wherein the transparent polymer film comprises a fluorinated polyimide having at least one aromatic ring; and graphitizing the fluorinated polyimide to form at least one layer of porous graphene on or within the transparent polymer film.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1A shows a schematic illustration for the synthesis of 3D porous graphene on a fluorinated polyimide film using a photothermal laser approach. The inset is the SEM image of the 3D porous graphene.

FIG. 1B shows an optical microscope image of the fPI-3DPG and a photograph of the fPI-3DPG nerve electrode inserted in a PCB board. The inset is the schematic of the cross-sectional porous graphene on the fPI film.

FIG. 1C shows experimental plots of an enhanced cathodic charge storage capacity (CSC) of fPI-3DPG neural electrodes compared to PI-3DPG.

FIG. 1D depicts mechanisms of the charge injection of the fPI-3DPG and PI-3DPG to stimulate a nerve.

FIG. 2A shows the correlation of the resistance with different laser powers. The scale bar of the inset images is 500 μm.

FIG. 2B shows an SEM image showing macro-scale porous structures in the fPI-3DPG.

FIG. 2C shows a cross-sectional SEM image of the fPI-3DPG.

FIG. 2D shows Raman spectra of fPI-3DPG synthesized at different laser powers.

FIG. 2E shows a statistical analysis of ratios of G and D peak intensities as a function of laser powers.

FIG. 2F shows average domain size along the a-axis (Lₐ) at different laser powers.

FIG. 3A shows impedance (Z) at 1 kHz for the described neural electrode.

FIG. 3B shows Bode magnitude plots of different electrode areas.

FIG. 3C shows Bode magnitude plots for certain electrode areas.

FIG. 3D shows Bode phase plots of fPI-3DPG and PI-3DPG electrodes.

FIG. 3E shows CV characterization of fPI-3DPG and PI-3DPG at a scan rate of 100 mV s⁻¹.

FIG. 3F shows CSC of 3DPG compared to single-layer graphene.

FIG. 3G shows voltage transient measurements of fPI-3DPG

FIG. 3H shows voltage transient measurements of PI-3DPG electrodes.

FIG. 3I shows the measurement of the charge injection limit of fPI-3DPG and PI-3DPG.

FIG. 4A shows a prolonged CV of the 3DPG, 1000 cycles at a scan rate of 100 mV s⁻¹, showing high stability of the electrode.

FIG. 4B shows a Bode magnitude plot of fPI-3DPG after prolonged pulse stimulation over 1 million cycles.

FIG. 4C shows impedance of the fPI-3DPG after cyclic bending tests over 10,000 cycles.

FIG. 4D shows electrical conductivity of the fPI-3DPG after cyclic bending tests over 10,000 cycles.

FIG. 5A shows a photograph of the fPI-3DPG electrodes under the anterior portion of the ventral cord in the lobster tail as well as the recording electrode close to the neural electrodes. The inset is the enlarged view of the fPI-3DPG electrodes under the anterior portion of the ventral cord in the lobster tail.

FIG. 5B shows a schematic of the ex vivo set up used for electrical stimulation studies.

FIG. 5C shows recorded combined artifact signals and compound action potentials during ex vivo stimulation by fPI-3DPG.

FIG. 5D shows an enlarged view of the combined stimulation artifact signal and compound action potential.

### DETAILED DESCRIPTION

### I. Neural Stimulation Device

In one aspect, the disclosed neural stimulation device comprises: a substrate; a transparent polymer film deposited on the substrate; and at least one layer of porous graphene on or within the transparent polymer film. In some aspects, the at least one layer of porous graphene has the following pore structure: macropores having an average pore size exceeding 50 nm; mesopores having an average pore size of 2-50 nm; micropores having an average pore size of 2 nm or less; and nanopores having an average pore size of less than 100 nm. In a further aspect, the nanopores have a BET specific surface area of at least 300 m²/g.

The substrate can be any suitable substrate, such as those commonly used as contacts for neural or electrical stimulation devices. With the integrated porous graphene, one or more of the substrates can function as a cathode or anode. The device can further feature components commonly used in neural stimulation devices such as electrical interconnects, e.g., silver interconnects, among others.

The transparent polymer film in some aspects can have an average thickness of 20-300 μm, e.g., 50-300 μm, 75-300 μm, 100-300 μm, 100-200 μm, 110-180 μm. In some aspects, the transparent polymer film can have an average thickness of 115-130 μm, e.g., 120 μm. As described below, in one aspect, the transparent polymer film can comprise a fluorinated polyimide, which can be used as a precursor to make the at least one layer of porous graphene on or within the transparent polymer film.

### A. Three-Dimensional Microporous Graphene

In one aspect, the disclosed 3D porous graphene (3DPG) is formed from flourinated polymides as described hereinafter and may be referred to as fluorinated polyimide-based highly microporous graphene (“fPI-3DPG”). In this disclosure, the properties and physical characteristics of fPI-3DPG are often compared with the properties and physical chatacteristics of 3DPG based on a non-fluorinated polyimide-based porous graphene (“PI-3DPG”). Both chemical structures are shown below in the Examples.

The nanopores of the fPI-3DPG in some aspects can provide for a high or even ultra high specific surface area, measured by the BET method which is known in the art. In some aspects, the nanopores of the porous graphene have a BET specific surface area of at least 300 m²/g. In a further aspect, the nanopores of the fPI-3DPG have a BET specific surface area of at least 400 m²/g. In a further aspect, the nanopores of the fPI-3DPG have a BET specific surface area of at least 500 m²/g. In a further aspect, the nanopores of the fPI-3DPG have a BET specific surface area of at least 600 m²/g. In a further aspect, the nanopores of the fPI-3DPG have a BET specific surface area of at least 700 m²/g. In a further aspect, the nanopores of the fPI-3DPG have a BET specific surface area of at least 800 m²/g. In a further aspect, the nanopores of the fPI-3DPG have a BET specific surface area of at least 900 m²/g. In a further aspect, the nanopores of the fPI-3DPG have a BET specific surface area of at least 1,000 m²/g. In a further aspect, the nanopores of the fPI-3DPG have a BET specific surface area of at least 1,200 m²/g. The upper limit for any of these threshold BET specific surface areas can vary, for example, 1,500 m²/g or in some aspects, 1,400 m²/g. In one aspect, the nanopores of the fPI-3DPG have a BET specific surface area of about 1,310 m²/g, “about” in this instance implying plus or minus 10 m²/g.

In a further aspect, the fPI-3DPG exhibits a Horvath-Kawazoe pore volume of at least 0.2 cm³/g. For example, the fPI-3DPG can exhibit a Horvath-Kawazoe pore volume of at least 0.25 cm³/g, at least 0.3 cm³/g, at least 0.35 cm³/g, at least 0.4 cm³/g, or at least 0.5 cm³/g. The upper limit for any of these pore volumes can vary, e.g., 0.8 cm³/g, 0.7 cm³/g, 0.6 cm³/g, or 0.55 cm³/g.

The described fPI-3DPG exhibits a high degree of graphitization as is evident from a number of characteristics. In one aspect, the porous graphene has a mean graphene interlayer spacing of 0.35-0.45 nm. In one specific embodiment, the interlayer spacing of the graphene is 0.39 nm. In some aspects the fPI-3DPG may include flourine atoms as a result of one exemplary method of making the fPI-3DPG described hereinbelow which uses fluorinated polymides as a precursor (e.g. graphitization is performed on fluorinated polymides). The flourine atoms may form C—F bonds and/or C—F₂ bonds in the 3DPG.

Thus, in one aspect, a disclosed embodiment of the 3D porous graphene may have a structure including one or more of the following pore structures: macropores having an average pore size exceeding 50 nm; mesopores having an average pore size of 2-50 nm; micropores having an average pore size of 2 nm or less; and nanopores having an average pore size of less than 100 nm. In addition, this embodiment may have a BET specific surface area of at least 300 m2/g or any of the BET surface areas described above. Further, this embodiment may include fluorine in the graphene, for example, fluorine disposed in individual sheets of the graphene and/or covalently bound to one or more carbon atoms of the graphene, e.g., comprising a C—F or C—F₂ bond). Additional details on the porous graphene are described in U.S. patent application Ser. No. 18/520,858, which is incorporated into this application by reference in its entirety, for its teachings of porous graphene and methods of making the porous graphene.

B. Method of Making the 3D Porous Graphene such as fPI-3DPG

Although the above-described graphene is not limited in scope to any particular production method, various multi-scale and porosity properties were surprisingly determined to be influenced by a method of manufacture. In some aspects, the porous graphene can be prepared by graphitizing a film of a fluorinated polyimide which has at least one aromatic ring, which can function or be integrated within the transparent polymer film. During graphitization, discharge of fluorine-based gas products, in addition to other gaseous products, was surprisingly discovered to result in a multi-scaled porous structure that is particularly amenable for electrically conductive applications such as neural stimulation devices.

In some aspects, the method allows for the construction of patterned or array style devices by graphitizing an already-filmed precursor polymer on demand, depending on the desired neural electrode application. In one aspect, graphitizing comprises irradiating the film with an infrared laser or other suitable types of lasers.

The fluorinated polyimide is not limiting provided it is capable of being graphitized, generally meaning it will have at least one aromatic ring, and provided it has at least one fluorine group. As discussed above, at least part of the fluorine will be released during graphitization to provide for unique porous structures and in turn optical properties. Some fluorine may remain in the layer of porous graphene as described above.

Non-limiting examples of such fluorinated polyimides include those having one of the following repeating units:

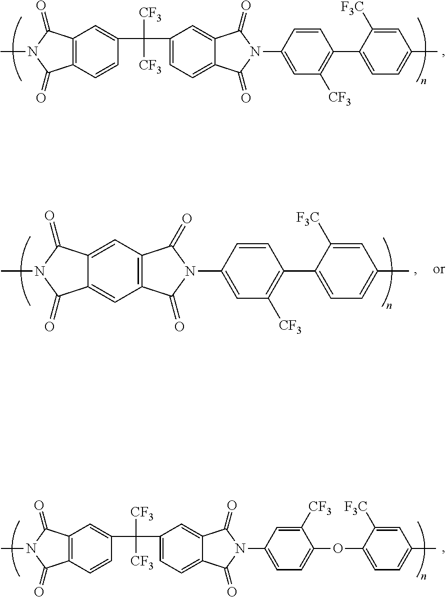

where each instance of n is independently an integer that is at least two. A variety of molecular weights beyond dimers are contemplated, typically only limited by the ability in some aspects to spin coat or otherwise solution coat the polyimide or a precursor thereof onto a substrate for imidization or graphitization. In one aspect, for example, the film of the fluorinated polyimide is prepared by thermal imidization of a precursor polyamic acid film.

Any suitable polyimide film thickness is contemplated, which in general will result in a film or graphene material of less thickness due to combustion of organic and other material, as well as organization of the graphene layers. In one aspect, the film of the fluorinated polyimide has an average thickness of 20-300 μm. A resulting graphene product or film can in some aspects have an average thickness of 10-180 μm. In one aspect, the graphene layer has a thickness of about 50 μm.

Graphitization of the fluorinated polyimide can be accomplished through a variety of contemplated methods. One example is laser irradiation of the fluorinated polyimide. For example, irradiating can be performed with a CO₂ infrared laser, e.g., having a wavelength (λ) of 10.6 μm. In a specific aspect, irradiating can be performed at 1-2 Watts, 1,000 laser pulses per inch (PPI), and at a speed of 3-4 inches per second, for example with a CO₂ infrared laser, e.g., having a wavelength (λ) of 10.6 μm.

### II. Method of Making and Using the Neural Stimulation Device

With the above principles in mind, the method for making the neural stimulation device generally comprises depositing the transparent polymer film onto the substrate, where the transparent polymer film comprises a fluorinated polyimide having at least one aromatic ring; and graphitizing the fluorinated polyimide to form at least one layer of porous graphene on or within the transparent polymer film. Graphitizing can be accomplished according to the conditions described above, e.g., irradiating the polyimide with a laser.

In one aspect, the method for stimulating a nerve of a subject comprises placing the neural stimulation device within sufficient proximity to the nerve to thereby stimulate the nerve. In some aspects, it is contemplated that the neural stimulation device will contact the nerve. In other aspects, the neural stimulation device may be localized to an area, e.g., an area of a subject's skin, surrounding the relevant nerve. Any subject is contemplated including mammals such as humans.

### III. Examples

The following examples further illustrate this disclosure. The scope of the disclosure and claims is not limited by the scope of the following examples.

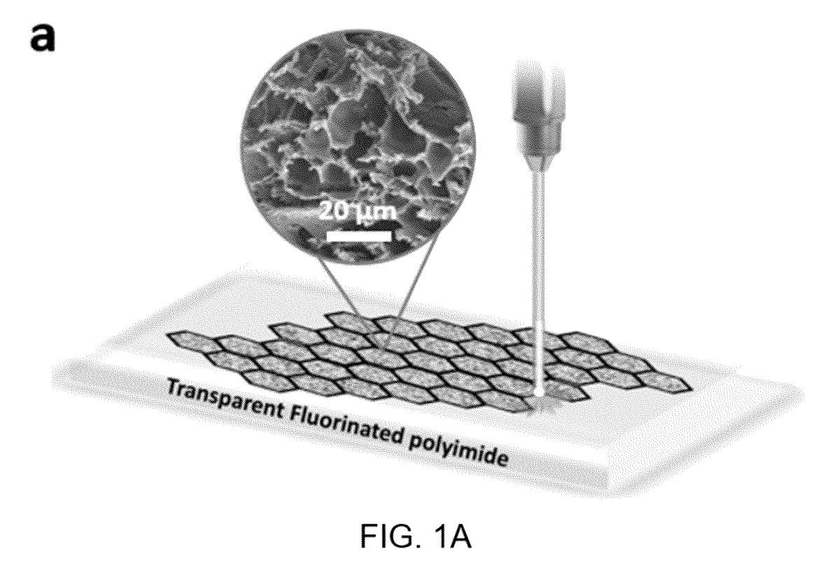

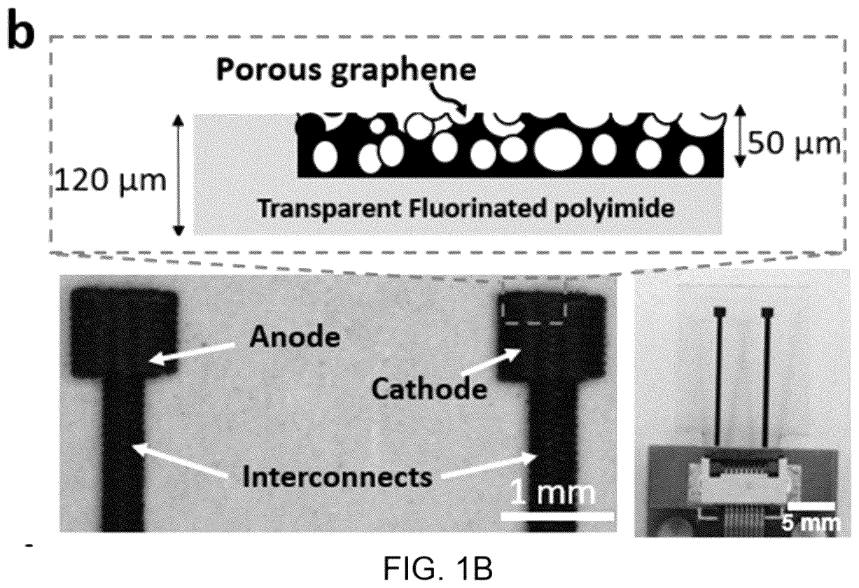

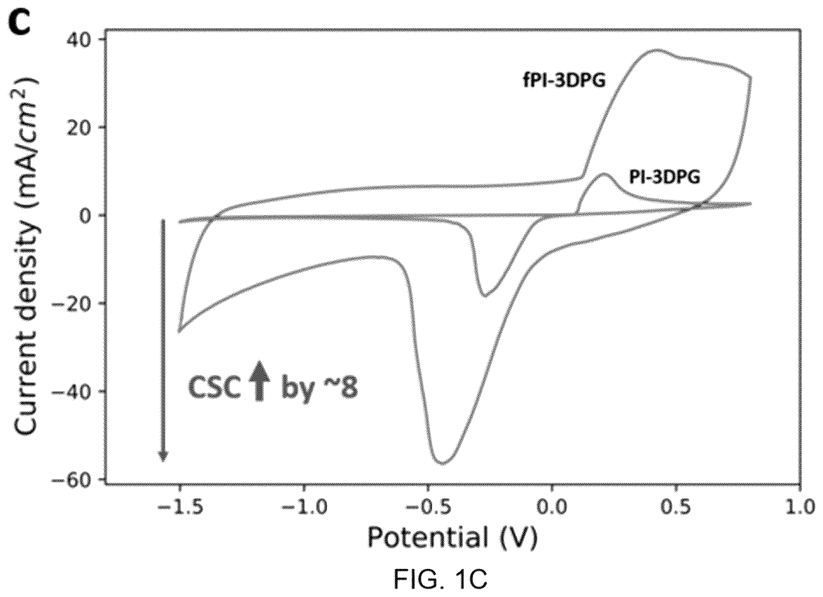

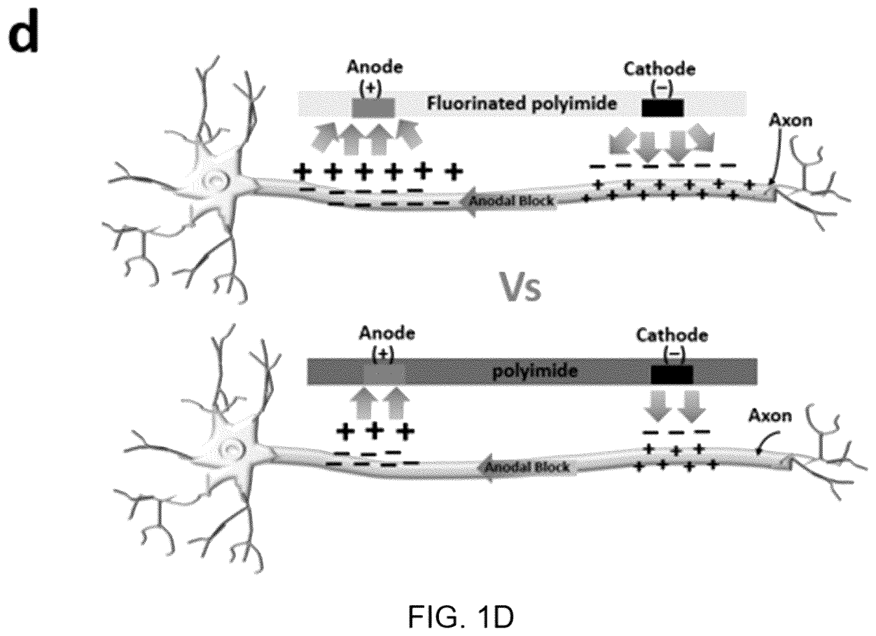

High-performance 3D micro-/nano-scale porous graphene-based (3DPG) neural electrodes were prepared which exhibited a high CSC and enhanced CIC for efficient neural stimulation. The 3DPG neural electrodes were fabricated using a scalable, rapid, and cost-effective direct laser scribing method from a fluorinated polyimide (fPI) precursor (FIG. 1A). The fluorinated polyimide precursor enabled the creation of highly microporous graphene structures with micro-/nano-scale pores through the release of fluorine-rich gaseous products under laser photothermal irradiation.

The fPI-3DPG was prepared from the following fluorinated polyimide.

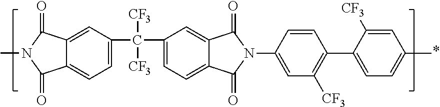

The comparative non-fluorinated polyimide (PI-3DPG) had the following structure.

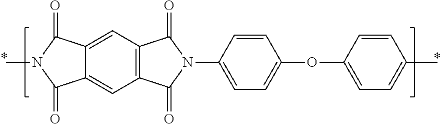

Microscope and photographic images of the 3DPG neural electrode derived from fPI (fPI-3DPG) are shown in FIG. 1B, displaying the bipolar electrodes and their integration into a printed circuit board (PCB) for conducting electrochemical measurements of CSC and CIC. The CSC of neural electrodes depends on the electrochemically active surface area of the electrode. The highly microporous structures of fPI-3DPG offer an enhanced specific surface area, allowing for greater charge storage on the electrode interface, resulting in improved CSC and better electrode performance with lower impedance. The structural characteristics of fPI-3DPG contribute to an eightfold enhancement in CSC and a twofold increase in CIC compared to PI-3DPG (FIG. 1C). fPI-3DPG neural electrodes, with their increased specific surface area and unique electrical properties such as high electron mobility, exhibit excellent electrochemical performance with high CIC and low impedance. The microporous structures of fPI-3DPG provide more active sites on the electrode surface, facilitating charge transfer between the electrode and surrounding tissue by reducing the travel distance for ions within the interconnected graphene pores and the tissue. This results in an increase in CIC compared PI-3DPG, which has fewer interconnected pores (FIG. 1D). 3D highly microporous graphenes with enhanced charge transfer efficiency and an eightfold increase in CSC compared to PI-3DPG have potential for use as a neural interface, enabling more effective and efficient stimulation of neural tissue.

C. Synthesis of fPI-3DPG Film

The generation of nanoscale pores in fluorine-doped polyimide (fPI) compared to plain polyimide (PI) plays a role in increasing the specific surface area of the disclosed materials, which is particularly valuable for applications like neural stimulation electrodes. The fPI film with a thickness of 120 μm was synthesized as follows. About 0.1 mol of 2,2′-bis(trifluoromethyl)-[1,1′-biphenyl]-4,4′-diamine (TFB) was dissolved in 326 ml of Anhydrous N,N-Dimethylacetamide (DMAc) in a 1 L 3-neck flat bottom type reaction flask equipped with a mechanical stirrer. Under the nitrogen atmosphere, the flask was placed in an ice bath, and 0.1 mol of 4,4′-(Hexafluoroisopropylidene) diphthalic anhydride was added to the solution. The mixture was stirred for an hour and stirred for 8 hours at room temperature. After the reaction, 20 wt % poly(amic) acids (PAA) solution was obtained. By adding the appropriate amount of DMAc, 12 wt % PAA solution was prepared, and 12.90 g of PAA solution was poured into a glass petri dish. The solution was heated under a vacuum to slowly evaporate the solvent and form the film. The film was imidized at 310° C. under a nitrogen atmosphere, and fluorinated polyimides film was fabricated. As a skilled artisan would understand, the above method for fabricating fluorinated polyimide films is not limited by particular process parameters such chemicals used, equipment types, temperatures, etc. Various adaptations and variations of the method may be implemented that would lead to fabricating the fluorinated polyimide films.

A pulsed infrared CO₂ laser with a wavelength of 10.6 μm was used to construct the highly microporous 3D graphene on fPI, employing different laser power settings under ambient conditions. Laser-induced graphene (LIG) forms when a high-powered laser heats carbon-containing fluorinated polyimide, creating a graphene-like structure with hexagonal carbon atoms. Simultaneously, the laser generates micro- and nano-interconnected porous structures, disrupting atomic bonds like carbon-carbon and carbon-fluorine. This process liberates carbon atoms that assemble into a distinctive hexagonal graphene lattice. LIG combines graphene networks with micro- and nano-scale pores. These pores, generated in fluorinated polyimide (fPI) through C—F bond cleavage and chemical reactions, increase surface area. This enhanced area benefits neural stimulation electrodes by improving contact with neural tissues, enhancing signal transmission efficiency, and making these materials suitable for neuroprosthetics and neural interfaces. It is understood that other laser types, wavelengths, laser powers, and pulse sequences may be used to fabricate the 3D porous graphene.

### D. Neural Electrode Device and Characterization

A 10.6 μm CO₂ laser engraving and cutting system (Universal, VLS2.30, Universal Laser Systems Inc.) was utilized for irradiating 120 μm-thick fluorinated polyimide films. 3D microporous graphene was synthesized at laser powers of 1.95 W, the scan speed of 3.5 in s⁻¹, and 1000 PPI. It is understood that other laser types, wavelengths, laser powers, and pulse sequences may be used to fabricate the 3D porous graphene.

Microporous graphene electrodes in various dimensions and geometries were patterned on an fPI film using a scalable, rapid, and one-step photothermal laser scribing approach with optimized laser setting parameter. Ag paste was printed on the interconnects and contact pads to increase the electrical conductivity of the device. After encapsulation of the interconnects with a thin layer of PDMS, the fabricated electrode was inserted in a printed circuit board (PCB) to connect the device to a potentiostat.

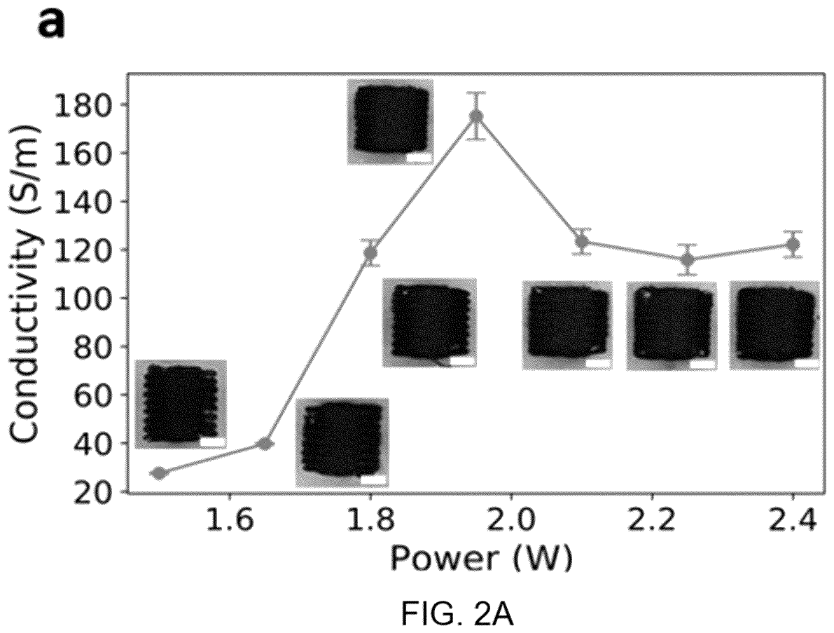

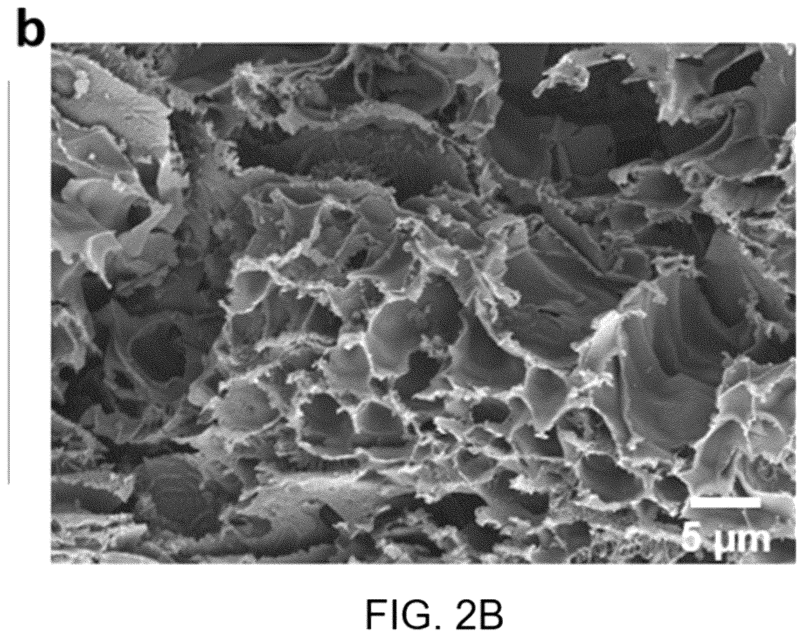

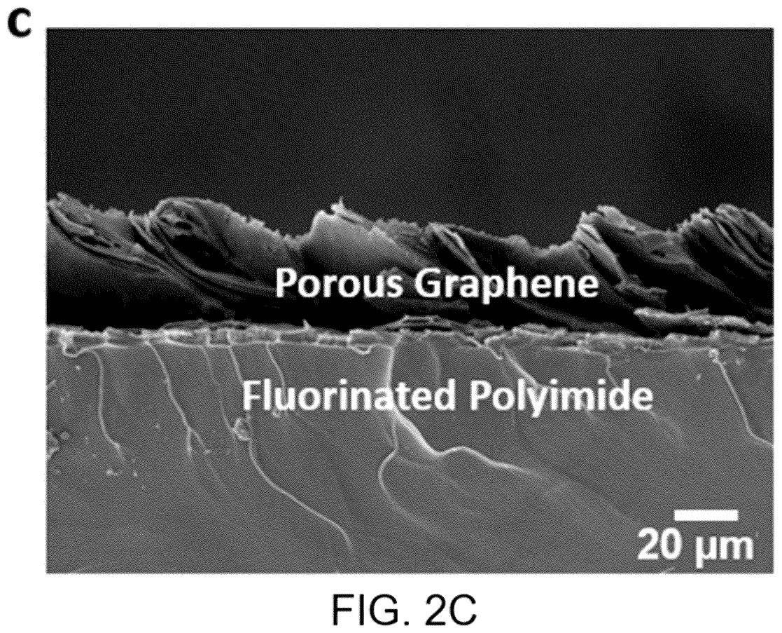

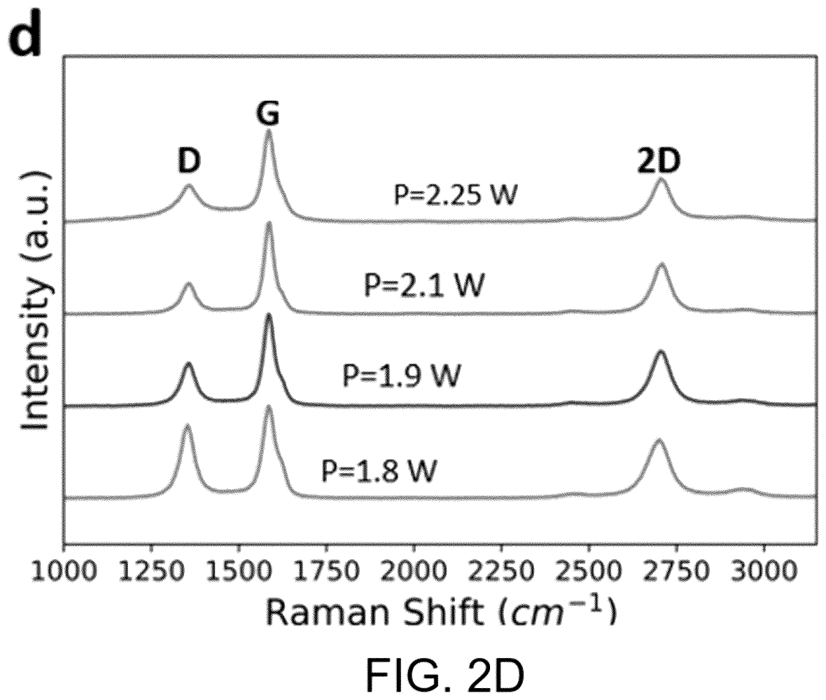

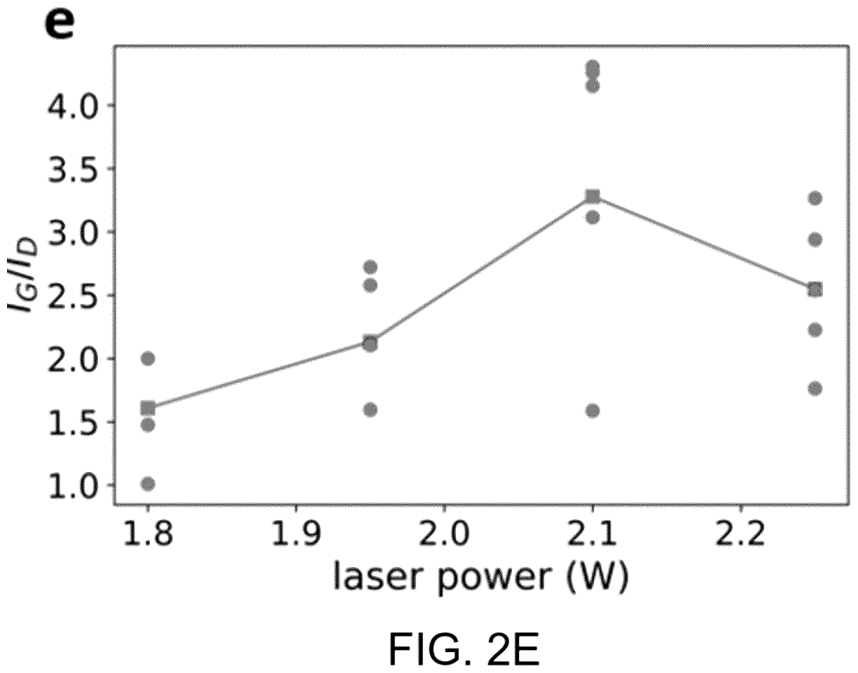

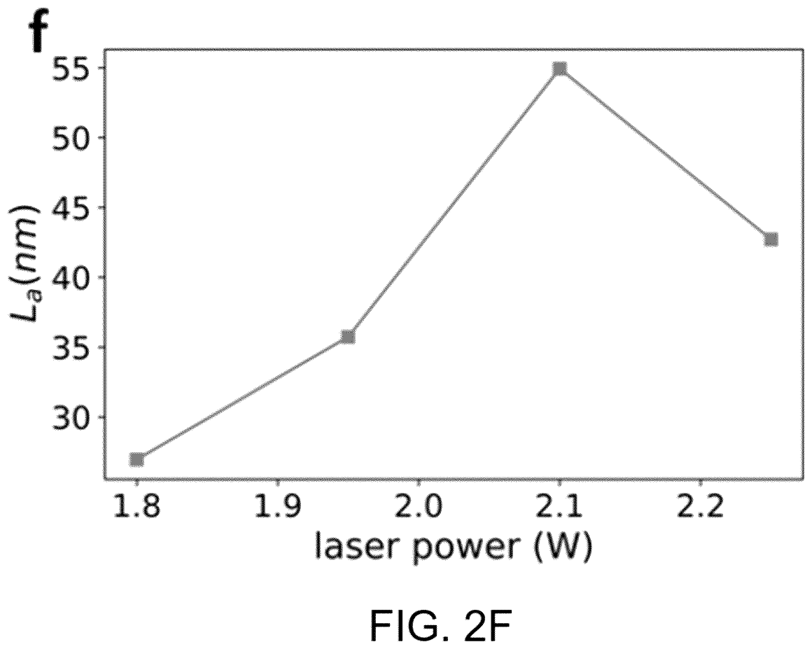

To evaluate the electrical properties of the fPI-3DPG neural electrode resulting from the laser manufacturing processing and to establish a property-processing relationship, the laser power within the range 1.5 W to 2.4 W was varied with a constant PPI of 1000 and a speed of 3.5 in s⁻¹. Resistance measurements were performed on fPI-3DPG fabricated at laser powers (Pₗₐₛₑᵣ) of 1.5, 1.65, 1.8, 1.95, 2.1, 2.25, and 2.4 W (FIG. 2A).

fPI-3DPG fabricated at Pₗₐₛₑᵣ=1.95 W exhibited the highest conductivity, indicating that 1.95 W represents an acceptable laser power setting. The morphology of the porous structures in fPI-3DPG fabricated at laser powers of 1.8, 1.95, 2.1 and 2.25 W was also evaluated using SEM images. fPI-3DPG synthesized at higher laser powers (2.1 and 2.25 W) displayed cracked graphene flakes structure, suggesting that elevated laser powers may have a detrimental effect on the quality of the porous nanostructure.

Furthermore, heterogeneous macropores with pore size distributions ranging from ˜1-10 μm in fPI-3DPG-1.95 W were observed (FIG. 2B). In addition to micro-scale pores, the fPI-3DPG, with a thickness of 50 μm (FIG. 2C), also retains rich nanosized pores, contributing to an increased specific surface area compared to PI-3DPG, which contains only micro-scale pores. The enhanced specific surface area and the presence of highly microporous structures provide for active sites on the electrode surface. This feature enhances the charge storage and charge transfer efficiency, ultimately leading to a larger CSC and CIC for neural stimulation. The results of the electrical analyses indicated acceptable laser parameters for creating highly microporous graphene are as follows: a laser power of 1.95 W, a laser speed of 3.5 inches per second, and PPI of 1000.

To confirm the optimal laser power graphitization for the transformation of amorphous carbon within fPI into highly ordered graphitic structures and ensure a high degree of graphitization, Raman spectroscopy on fPI-3DPG fabricated at different laser power levels was conducted, ranging from 1.8 W to 2.25 W, with maintaining a constant PPI of 1000 and a speed of 3.5 in s⁻¹ (FIG. 2D). The statistical analysis of I_{G}/I_{D} and the corresponding crystalline size (Lₐ) along the a-axis of graphitic structures are shown in FIGS. 2E and 2F. The achievement of a high degree of graphitization translates to enhanced electron mobility, thereby increasing the electron transfer rate for charge injection during neural stimulation. When 3DPG was fabricated using a laser power of 2.1 W or higher, flaking and an inhomogeneous structure were observed. In contrast, fPI-3DPG fabricated at Pₗₐₛₑᵣ=1.95 W showed a high degree of graphitization, characterized by a larger and consistent I_{G}/I_{D} ratio of 2.2 and crystallinity with a Lₐ of 36 nm. As the laser power increased, there was a corresponding decrease in the intensity of the D peak, while the intensity of the G peak exhibited a continuous augmentation. This increase continued until structural damage manifested itself, evident through dispersal of G-to-D peak intensity ratio at Pₗₐₛₑᵣ=2.1 W, as shown in FIG. 2E.

The escalating G peak intensity in comparison to the D peak suggests a greater occurrence of in-plane stretching vibrations within sp2-bonded carbons, indicative of an increased presence of π bonded C═C networks and a decrease in sp2 bonding disruptions. Furthermore, the presence of the 2D peak serves as an indicator of the number of layers within the porous graphene, originating from a dual resonance-enhanced two-phonon lateral vibrational process. The 2D-to-G peak intensity ratio (I_{2D}/I_{G} ratio) of GNFs slightly showed slight decreases with increasing laser power as shown in the Table below.

| Laser power (W) | I_{2D}/I_{G} | I_{D}/I_{G} | I_{G}/I_{D} |
|---|---|---|---|
| 1.8 | 0.66 | 0.63 | 1.61 |
| 1.95 | 0.59 | 0.48 | 2.1 |
| 2.1 | 0.55 | 0.36 | 3.28 |
| 2.25 | 0.47 | 0.42 | 2.55 |

An I_{2D}/I_{G} ratio ranging from 0.66 to 0.47 indicates single-layer graphene synthesis during photothermal laser process of fPI. The Raman spectra of the graphitic structures in both fPI-3DPG and PI-3DPG, fabricated at Pₗₐₛₑᵣ=1.95 and 3.6 W, respectively, showed three main peaks at ˜1350, 1580, and 2700 cm⁻¹, representing D, G, and 2D peaks, respectively. The presence of these peaks in the Raman spectra indicates the formation of graphitic structure through photothermal laser irradiation.

To further validate the sp2 hybridization state of carbon, X-ray photoelectron spectroscopy (XPS) was employed. The XPS spectra revealed that the primary C1s characteristic peak is centered at 284.2 eV, with negligible characteristic peaks for O1s and F1s. This observation supports the prevalence of C—C bonds in the porous graphene, further confirming the formation of a graphitic structure. The Raman and XPS spectra results corroborated the successful transformation of amorphous carbon within the fPI into highly graphitized structures using the photothermal laser manufacturing technique. This is supported by the presence of three distinct characteristic peaks (D, G, and 2D), a substantial crystalline size of 36 nm, and the formation of sp2 carbon.

The successful implementation of neural electrodes, particularly in head and neck surgery, relies on surgical techniques and accurate nerve identification to prevent damage and post-operative complications. Consequently, the transparency of the neural electrode plays a role. To demonstrate the potential of the fPI-3DPG neural electrode in imaging applications, the optical characteristics of fPI and PI films were investigated using UV-Vis spectroscopy. fPI exhibited a high transmittance rate of 90% within the 400-800 nm wavelength range. This characteristic not only makes fPI a good choice for enhancing neuroimaging systems but also holds promise for optimizing optogenetic procedures. In contrast, PI revealed non-transparency within the wavelength range of 400-500 nm, coupled with a low transmittance percentage (T) falling in the range of 0<T %<65% within the wavelength range 500-600 nm. This opacity not only obstructs the surgeon's field of view but also results in the inadvertent loss of information during nerve tissue surgeries. The results of UV-Vis confirm the potential of fPI-3DPG for integrating imaging-based identification of peripheral nerve with simultaneous neural stimulation.

### E. Cathodic Charge Storage and Charge Injection Capacity Studies

To determine the cathodic charge storage capacity and charge injection capacity of the fPI-3DPG neural electrode and to evaluate its electrochemical performance for neural stimulation, a series of electrochemical tests were conducted, including electrochemical impedance spectroscopy (EIS), cyclic voltammetry (CV), and voltage transient (VT) measurements. A high electrical impedance at the interface between the electrode and the nerve can increase the risk of electrode and nerve damage during neural stimulation since it requires higher stimulation voltage. The electrical impedance is influenced by the dimensions of micro-scale electrodes. Therefore, square fPI-3DPG electrodes were fabricated with different widths (600, 900, and 1200 m) to confirm the effect of the electrode size on the device impedance. Detailed EIS measurements were performed for each device, and the impedance of each electrode was compared at frequency of 1 kHz.

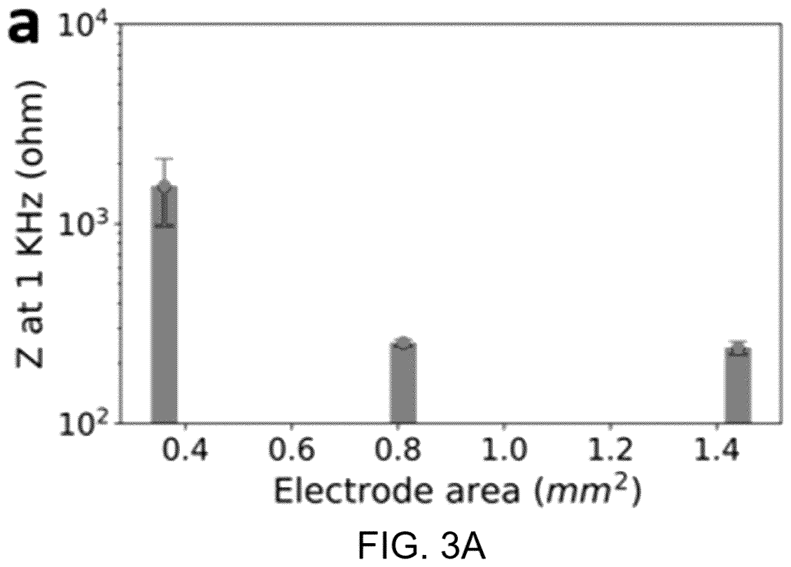

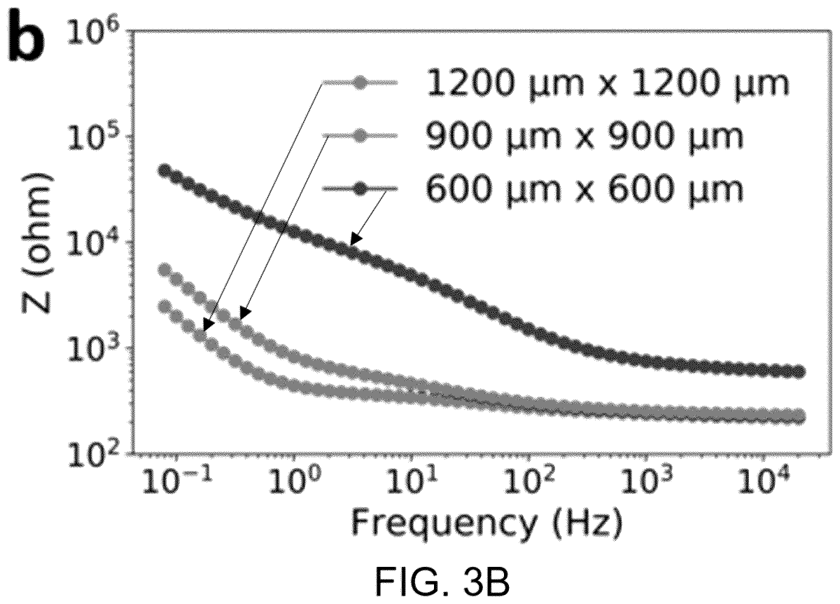

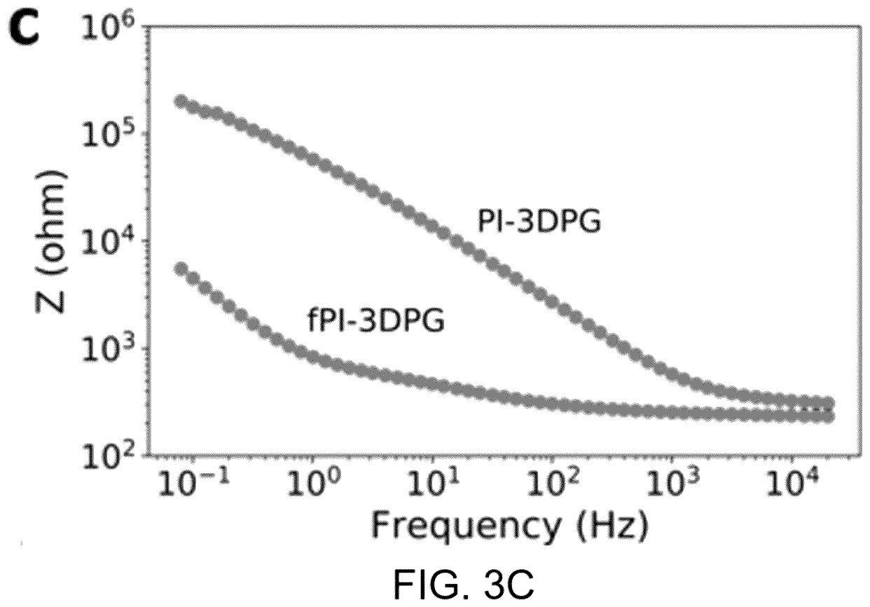

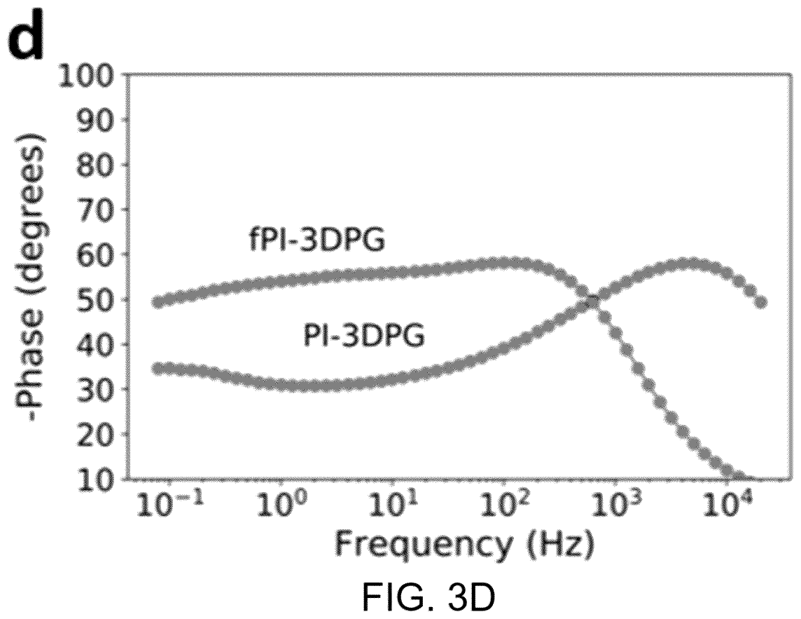

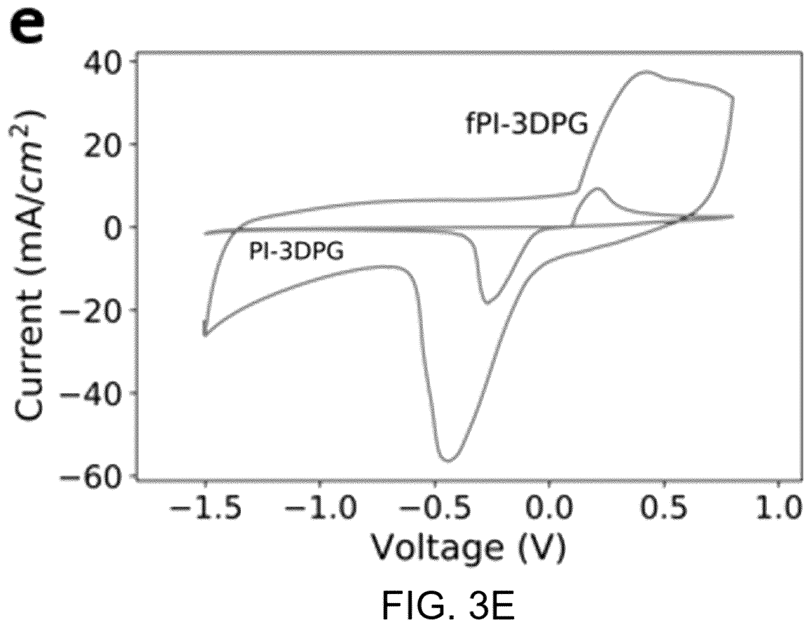

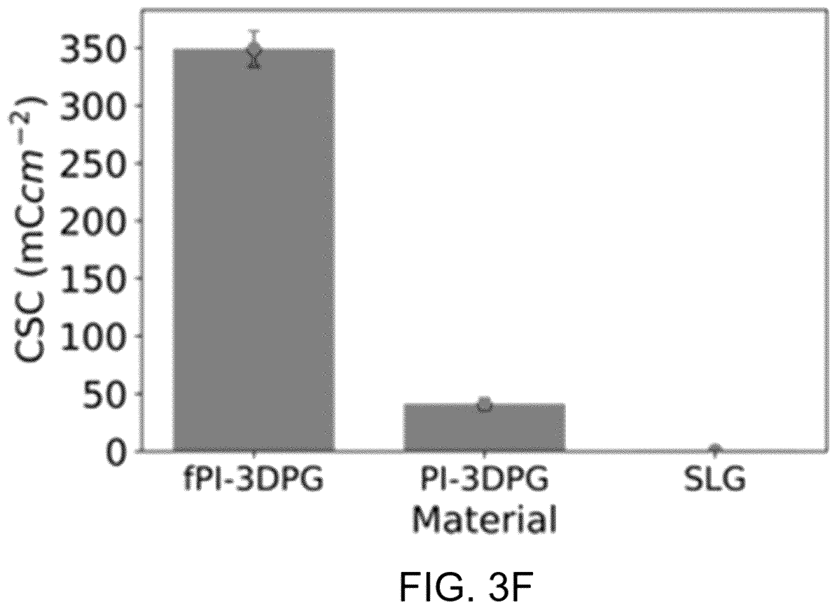

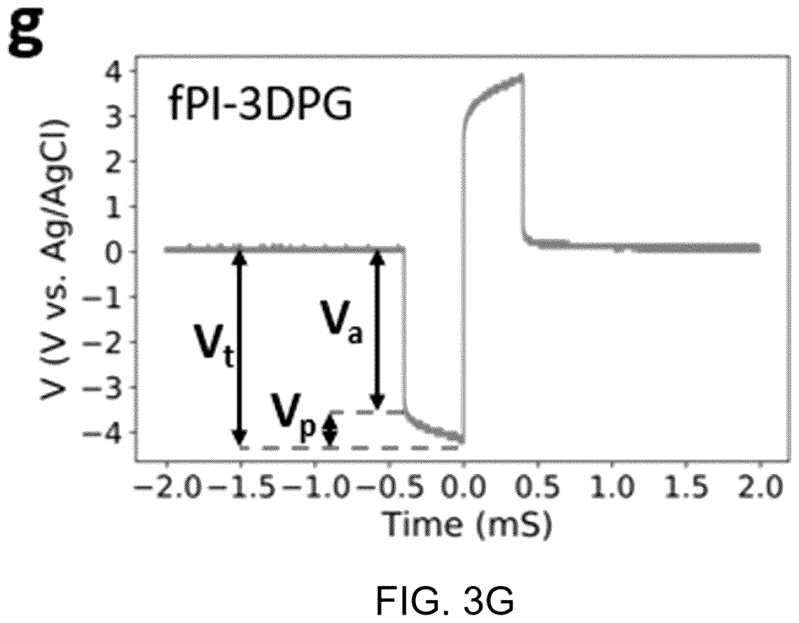

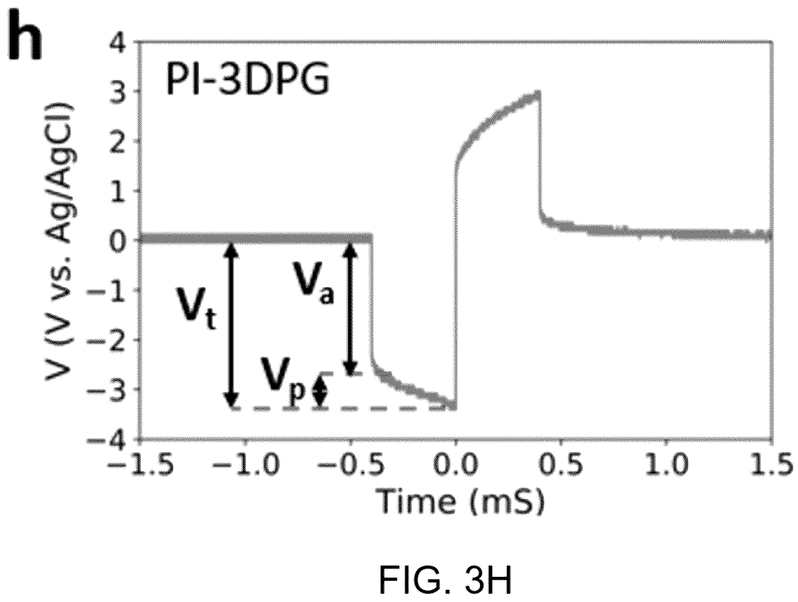

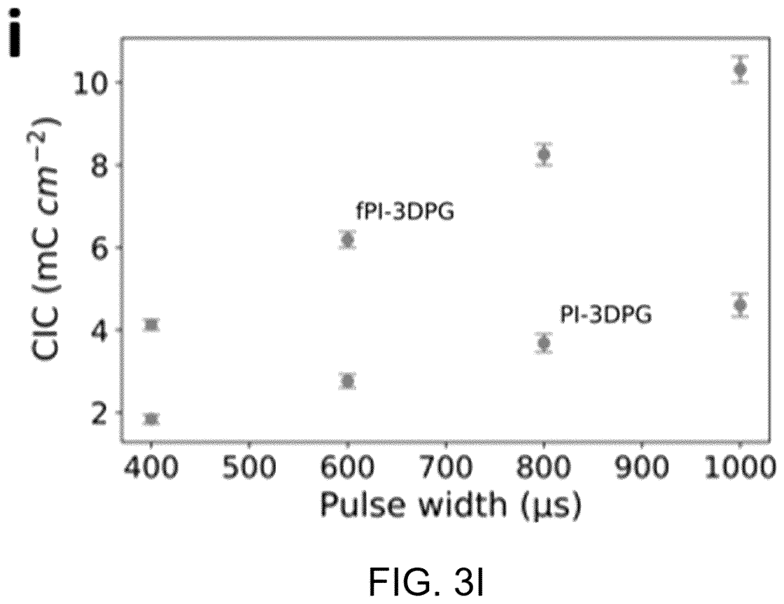

The EIS analysis showed the average impedances of 1.55±0.57, 0.25±0.009, and 0.24±0.017 KΩ for electrodes with surface areas of 0.36, 0.81, and 1.44 mm², respectively (FIG. 3A). The decrease in impedance with a larger electrode surface area emphasizes the complex interdependence of impedance values and the extent of the active electrode surface. This finding has implications for optimizing electrode design and performance, indicating that maximizing surface area could enhance electrical properties and potentially improve the charge transfer efficiency during neural stimulation.

The Bode plot displayed in FIG. 3B, illustrating impedance (Z) for different electrode areas across a measured frequency range (0.1 Hz-20 kHz), shows that devices with low surface area (0.36 mm²) exhibit higher impedances compared to those with larger surface areas. All the electrochemical characterizations on the fabricated devices were performed with a surface area of 0.81 mm², unless otherwise indicated. The impedance of the fPI-3DPG electrode was an order of magnitude lower than that of PI-3DPG electrode within the frequency range of 0.1-500 Hz (FIG. 3C). The specific impedance at 1 kHz, which is a benchmark frequency of neural electrodes, was over 50% lower than that of PI-3DPG (fPI=3DPG=254Ω vs PI-3DPG=575Ω). This reduction in impedance of fPI-3DPG compared to PI-3DPG can be attributed to the enhanced accessible specific surface area of fPI-3DPG, which facilitates more efficient electrical charge transfer between the electrode and the surrounding tissue.

The fPI-3DPG device exhibited a more negative phase angle than the PI-3DPG device within the frequency range of 0.1-1000, indicating a more capacitive charge transfer behavior of the fPI-3DPG (FIG. 3D). A higher capacitive charge transfer is desirable for neural stimulation, as it allows for precise, efficient, and controlled delivery of electrical pulses to modulate neural activity while minimizing tissue damage and reducing power consumption. With its reduced impedance of 254Ω at 1 kHz and enhanced capacitive charge transfer behavior, fPI-3DPG demonstrates potential for the development of high-quality neural electrodes capable of stimulating nerves at low stimulus amplitudes.

To compare the performance of the fPI-3DPG neural electrode in storing and delivering electrical charge with that of PI-3DPG, CV measurements were conducted in phosphate-buffered saline (PBS) and assessed the charge storage capacity and charge injection capacity of both neural electrodes. The CV curve of fPI-3DPG exhibited a higher current density than that of PI-3DPG, confirming the larger capacitance of fPI-3DPG (FIG. 3E). The CSC, a desirable property of electrode used for biological stimulation, was quantified from the CV curves by integrating the area under cathodic currents divided by scan rate. The calculated cathodic CSC of the fPI-3DPG electrode was 362.4 mC cm⁻², which is eight times higher than that of the PI-3DPG electrode. Moreover, the CSC of the fPI-3DPG is more than two orders of magnitude higher than that of single-layer graphene (SLG) (FIG. 3F), and superior to carbon-based and metal-based electrode materials reported to date; a detailed comparison is presented in the Table below.

| | | Specific | Charge | Charge |
|---|---|---|---|---|
| | Surface | impedance | storage | injection |
| | area | at 1 KHz | capacity | Limit |
| Material^{a)} | [μm²] | [MΩ μm²] | [mC cm⁻²] | [mC cm⁻²] |
| fPI-3DPG | 900000 | 208 | 362.4 | 4.4 |
| PI-3DPG | 900000 | 366 | 48.1 | 2 |
| Doped LIG | 625000 | 324 | 50 | 3.1 |
| SLG | 2500 | 7500 | 0.7 | NA |
| Doped-SLG | 2500 | 1500 | 1.9 | NA |
| Graphene | 31416 | 3141 | 0.09 | 0.06 |
| CNTs | 50000 | 100 | NA | 1.6 |
| CNT fiber | 1450 | 20.44 | 372 | 6.52 |
| Porous diamond | 314 | 53.69 | 10 | 3 |
| PEDOT:PSS | 4500 | 27 | 123 | 2.9 |
| PEDOT-CNT | 2830 | 42.45 | 6 | 1.25 |
| GF-PC-40 | 169 | 28 | 200 | 4.7 |
| GF-PC-20 | 749 | 8.7 | 798 | 8.9 |
| Pt-grass | 1256 | 125 | NA | 0.3 |
| PtIr | 4500 | 405 | 8 | 0.13 |
| Porous Pt | 2000 | 50 | 18.46 | 0.5 |
| Porous TiN | 2830 | 155.65 | 5 | 0.7 |
| IrOx | 4500 | NA | 0.2 | NA |

LIG: laser-induced graphene; SLG: single-layer graphene; CNT: carbon nanotube; PEDOT: poly(3,4-ethylenedioxythiophene); PSS: polystyrenesulfonate; GF-PC: graphene-fiber-parylene-C; PtIr: platinum iridium; TiN: titanium nitride; IrOx: iridium oxide.

This enhancement in CSC can be attributed to the structural characteristics of fPI-3DPG, which include highly microporous structures. These microporous features create a larger electrode surface area, providing more space for the storage of ions and electrons, leading to a higher CSC. In neural stimulation, where controlled delivery of electrical charges to modulate neural activity is essential, a larger surface area translates to a higher charge injection capacity. This increased CIC allows the electrode to effectively deliver a wider range of electrical currents, meeting the diverse requirements of neural stimulation protocols while maintaining safety. The CIC of a neural stimulation electrode depends on various factors, including surface area and surface chemistry. Larger surface areas promote more efficient charge transfer, and surface chemistry influences the formation of a stable interface between the electrode and tissue.

To determine CIC, VT measurements were conducted to determine the charge limit required to polarize the electrode-electrolyte interface within a stimulation pulse. This determination ensures that the CIC remains below the thresholds for water reduction or oxidation potential, preventing undesirable chemical reactions at the interface during neural stimulation, which could lead to electrode damage or unsafe conditions. Symmetric biphasic current pulses width of 400 μs were applied to both fPI-3DPG and PI-3DPG electrode. The polarization voltage (Vₚ) of the fPI-3DPG electrodes was determined to be −0.8 V by subtracting the access voltage (Vₐ=−3.68 V) at the onset of the pulse from the maximum negative voltage transient (Vₜ=−4.48 V) (FIG. 3G). The CIC of the fPI-3DPG was quantified at Vₚ=0.8 V to be 4.13±0.13 mC cm⁻² according to CIC=I.P_{w}/A, where I is the amplitude of the stimulation current (0.01 A), P_{w} is the pulse width of the stimulation signal (400 μs), and A is the surface area of the electrode (˜0.0009 cm²). The CIC of the PI-3DPG was also calculated at Vₚ=0.6 V to be 1.84±0.11 mC cm⁻² (FIG. 3H). The two-fold enhancement in CIC of fPI-3DPG compared to PI-3DPG is attributed to the microporous structures of fPI-3DPG, which provide more active sites on the electrode surface for enhanced charge transfer between the electrode and surrounding tissue. The resulting CIC of the fPI-3DPG were significantly enhanced for all pulse widths tested, showing a superior CIC of 10.32±0.32 mC cm⁻² for fPI-3DPG at the pulse width of 1 ms (FIG. 3I). The electrochemical performance of the fPI-3DPG neural electrode, as implied by CSC=362.4 mC cm⁻² and CIC=10.32±0.32 mC cm⁻², which surpass CSC and CIC of PI-3DPG by a factor of eight and two, respectively, highlights its efficiency in storing and delivering electrical charge. This capability holds potential to enhance the effectiveness and safety of neural stimulation.

### F. Flexibility and Durability Studies

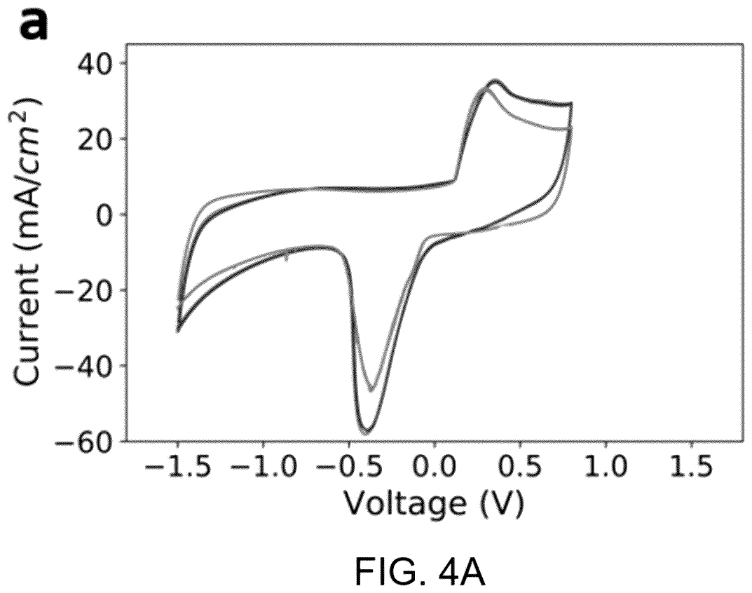

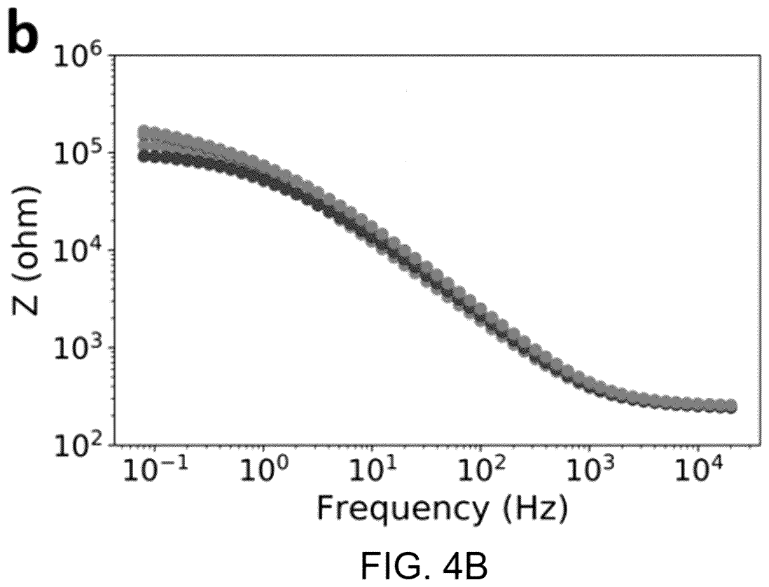

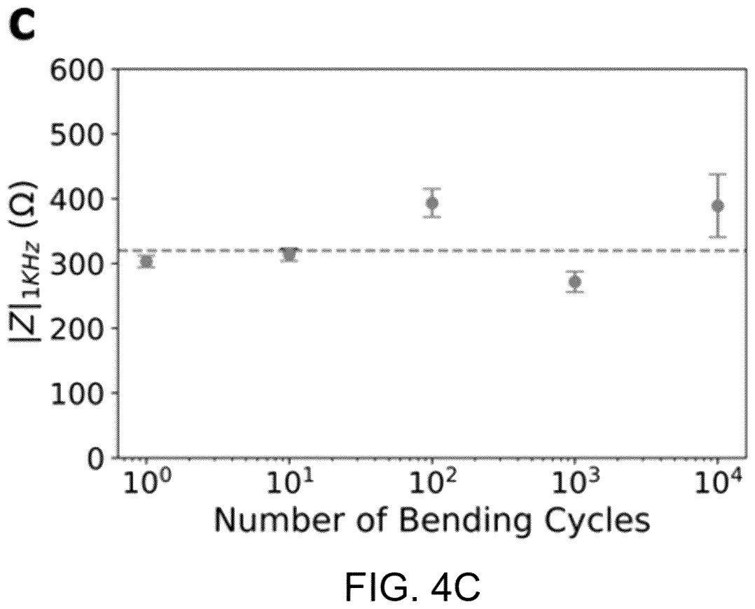

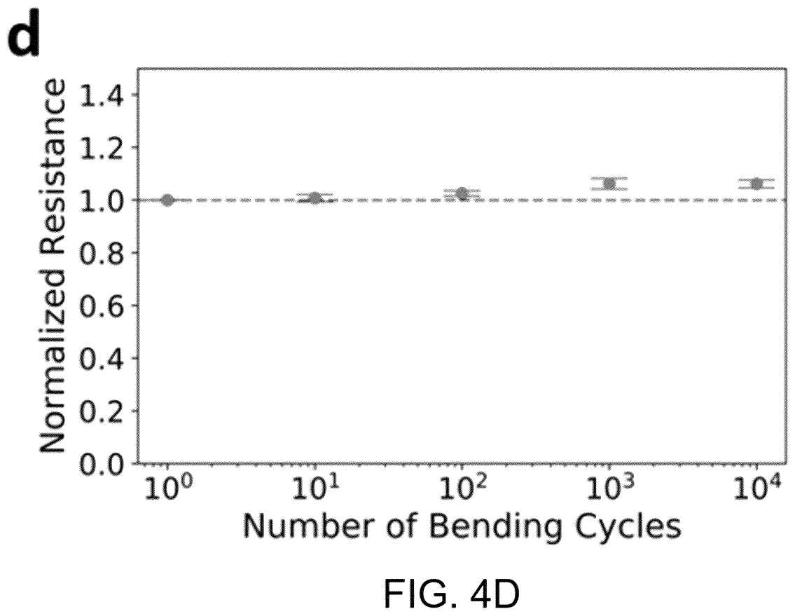

To evaluate the flexibility and durability of the fPI-3DPG electrode, both electrical and electrochemical characterizations were performed following 10000 cyclic bending tests, prolonged CV, and pulse stimulation measurements. This evaluation was useful, as implanted neural electrodes can be negatively altered by material degradation and the delamination of insulator coatings over time. The CV curves displayed in FIG. 4A confirm that there was no degradation in the electrochemical performance of the fPI-3DPG even after undergoing extended stability testing (up to 1,000 cycles). Furthermore, stimulation performance and cycling endurance of the fPI-3DPG was tested through subjecting fPI-3DPG electrodes to continuous stimulation by applying 1 V pulse voltage with 500 μs pulse width up to 10⁶ stimulation cycles. The impedance of the fPI-3DPG electrode exhibited negligible change after injecting charge over one million cycles, indicating the high stability of the electrode up to 1,000,000 cycles (FIG. 4B).

The mechanical robustness of the fPI-3DPG electrode was explored because the strong mechanical flexibility of the nerve electrode is important to conformably be integrated on the curved surface of the tissue. The consistent impedance (FIG. 4C) and resistance (FIG. 4D) of the fPI-3DPG, with minimal change under bending strains (ε_{b}=4.5%.) up to 10,000 cycles, verifies the mechanical robustness of fPI-3DPG. The durability demonstrated by the flexible and robust fPI-3DPG electrode, under conditions of 10,000 cycles of bending, 1,000 cycles of voltammetry measurements, and 1,000,000 cycles of pulse stimulation, carries implications for its potential impact in long-term neural applications. This durability ensures the sustained and reliable performance of the electrode, suggesting its suitability for extended use in neural interfaces, thereby advancing the reliability and longevity of neural stimulation technologies.

### G. Electrical Stimulation Studies

The Gamry Reference 1010B potentiostat was connected in the standard three-electrode configuration. All measurements were conducted using a laser-generated 3D porous graphene counter electrode and an isolated silver/silver chloride (Ag/AgCl) reference electrode immersed in 0.01 M Phosphate buffered saline (PBS, pH 7.4, Sigma-Aldrich) at room temperature. EIS measurements were taken between 0.1 Hz to 20 kHz using 10 mV RMS AC voltage. For CV tests, the potential of the working electrode was swept five times across the potential window between −1.5 and 0.8 V at the scan rate of 100 mV/s. Each device was tested for 5 cycles, and the cathodic charge storage capacity was calculated from the time integral of the cathodic current density of the last cycle within the potential window and divided by scan rate.

The experiment was conducted at room temperature in the same three-electrode configuration and electrolyte for EIS and CV. A symmetric charged-balanced, cathodic first, biphasic current pulses were applied at 200, 400, 600, and 800 μs pulse widths using a stimulator (Model 2100 Isolated Pulse Stimulator). Current amplitudes were incrementally increased until the polarization voltage measured with an oscilloscope (MSO 2002B, Tektronix) reached to Vₚ=−0.8 V.

A lobster with weight range of 600-700 g (Hmart, USA) were anesthetized and the tail was humanely cut off. fPI-3DPG electrodes (1 mm×1 mm) was placed under the anterior portion of the ventral cord in the lobster tail and a biphasic 0.9 mA current pulse, at constant frequency of 1 Hz, and pulse width of 400 μs were delivered from our fPI-3DPG neural electrodes to the nerve using a stimulator (Model 2100 Isolated Pulse Stimulator). The ground wire, Ag/AgCl reference electrode, was placed in the lobster near the tail. A recording electrode was a pair of stainless-steel electrodes bundled together with 3 mm distance from fPI-3DPG cathode. Recorded signals were collected with band-pass filter 250 Hz to 7.5 kHz for noise removal using 128-Channel Neural Signal Processor (Blackrock Microsystems Cerebus System). Ex vivo performance was characterized by recording artifact signal, action potential, and contraction of muscles.

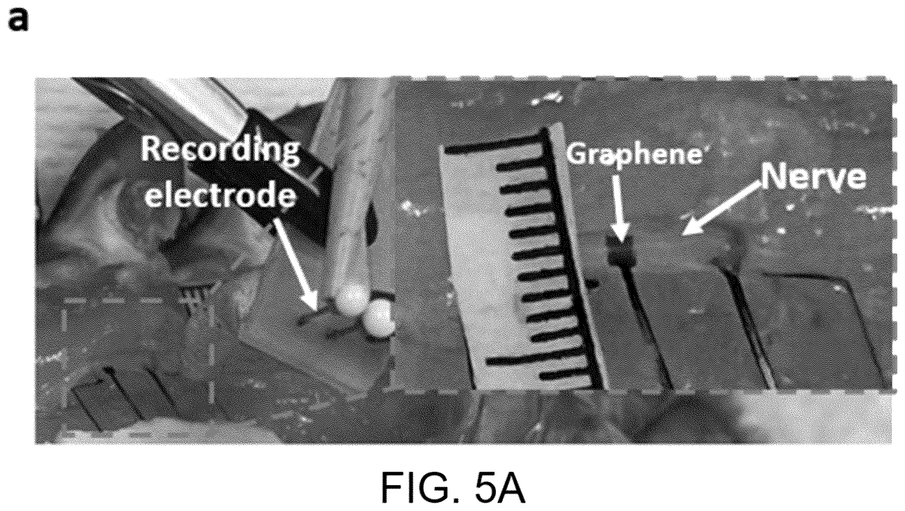

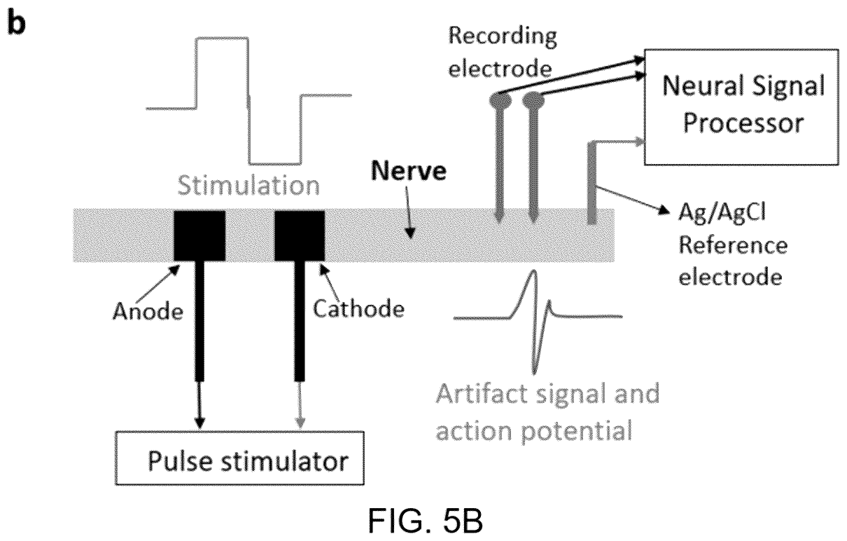

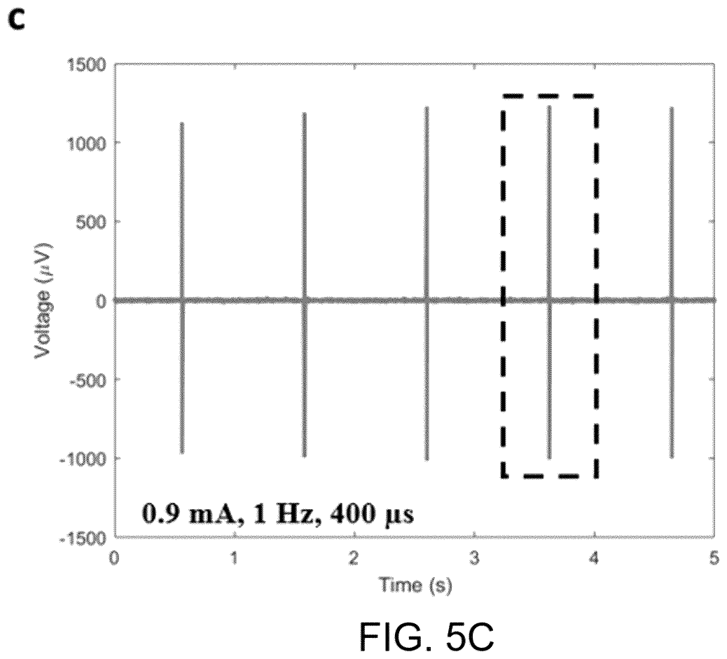

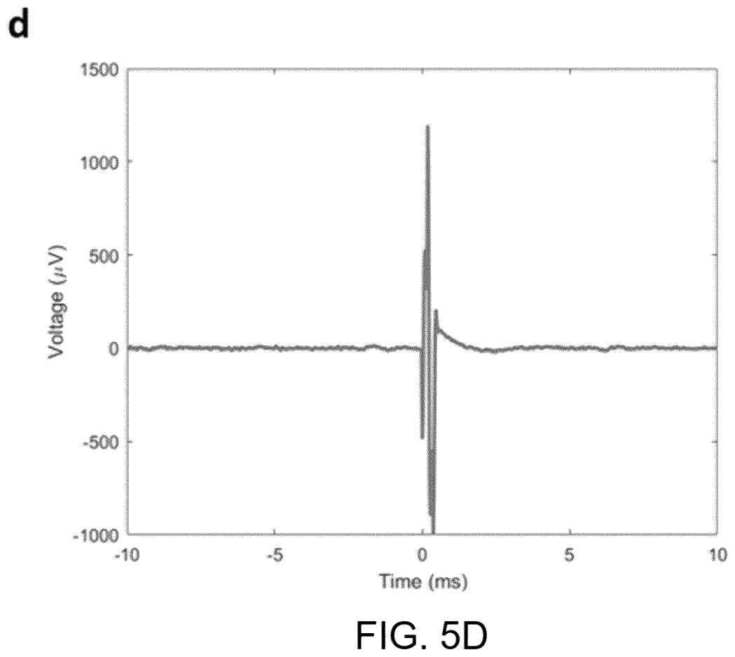

To provide a proof-of-concept for neural stimulation using the fPI-3DPG neural electrodes, an ex vivo testing was performed involving the electrical stimulation of a lobster tail. In this experiment, fPI-LIG electrodes were placed beneath the anterior portion of the ventral cord in the lobster tail, as shown in FIG. 5A. A series of continuous biphasic 900 μA current, a constant frequency of 1 Hz, and a pulse width of 400 μs were delivered to the nerve through the fPI-3DPG neural interface, thus stimulating the nerve through the fPI-3DPG neural interface.

The experimental setup for neural stimulation and potential signal recording is schematically illustrated in FIG. 5B. Electrical activity recorded during ex vivo is shown in FIG. 5C, illustrating continuous stimulus artifact signals. When the nerve was electrically stimulated, compound action potentials (CAP) propagated along the nerve, eliciting muscle contractions. FIG. 5D shows an enlarged view of the combined stimulation artifact signal and compound CAP in the lobster tail, indicating that a current pulse was injected to the nerve through the fPI-3DPG neural interface, and a recording electrode collected both the artifact signal and the CAP. This demonstration shows the capability of the fPI-3DPG electrodes to be used as neural interfaces for electrical stimulation of peripheral nerves in various neural stimulation applications.

These Examples present high-performance 3D highly microporous-structured graphene-based neural electrodes designed for efficient neural stimulation using one-step laser photothermal manufacturing technique. These structures are distinguished by micro- and nano-scale pores, achieved through the liberation of fluorine-rich gaseous byproducts during laser photothermal irradiation. The resulting fPI-3DPG neural electrode offers an increased electrochemical active surface area and unique electrical properties, and high electron mobility. fPI-3DPG also demonstrates excellent electrochemical performance, with a high CSC of 362.4 mC cm⁻² and a CIC of 10.32 mC cm².

Compared to the PI-3DPG electrode, fPI-3DPG exhibits an eightfold increase in CSC and a twofold increase in CIC. The CSC of the fPI-3DPG electrode surpasses that of monolayer graphene and metal-based electrodes by more than two orders of magnitude. The superior CSC of fPI-3DPG is likely attributable to its integrated microporous structure and high specific surface area, facilitating efficient charge transfer and subsequent enhancement in charge accumulation. The fPI-3DPG electrode also demonstrates high mechanical flexibility, reliability, and durability, enduring up to 10,000 cyclic bends, 10⁶ stimulation cycles, and 1000 continuous CV cycles. The effectiveness of the fPI-3DPG neural electrodes was further demonstrated through ex vivo electrical stimulation of lobster tail nerve, resulting in visible lobster muscle contractions. Finally, the synergistic effects of enhanced CSC and CIC of fPI-3DPG, along with the high transparency of fPI film present a future potential of the fPI-3DPG neural electrode for integrating imaging-based identification of peripheral nerves with simultaneous highly efficient neural stimulation for applications in optogenetics and neuroimaging.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

What is claimed is:

1. A neural stimulation device, comprising:
a) a substrate;
b) a transparent polymer film deposited on the substrate; and
c) at least one layer of porous graphene on or within the transparent polymer film;
wherein the at least one layer of porous graphene has the following pore structure:
i) macropores having an average pore size exceeding 50 nm;
ii) mesopores having an average pore size of 2-50 nm;
iii) micropores having an average pore size of 2 nm or less; and
iv) nanopores having an average pore size of less than 100 nm;
wherein the nanopores have a BET specific surface area of at least 300 m²/g.

2. The neural stimulation device of claim 1, wherein the nanopores have a BET specific surface area of 300-1500 m²/g.

3. The neural stimulation device of claim 1, wherein the at least one layer of porous graphene exhibits a Horvath-Kawazoe pore volume of at least 0.2 cm³/g.

4. The neural stimulation device of claim 1, wherein the at least one layer of porous graphene exhibits a Horvath-Kawazoe pore volume of 0.2-0.8 cm³/g.

5. The neural stimulation device of claim 1, wherein the at least one layer of porous graphene has a mean graphene interlayer spacing of 0.35-0.45 nm.

6. The neural stimulation device of claim 1, wherein the transparent polymer film comprises a fluorinated polyimide having at least one aromatic ring.

7. The neural stimulation device of claim 6, wherein the at least one layer of porous graphene is prepared by graphitizing the fluorinated polyimide.

8. The neural stimulation device of claim 7, wherein graphitizing comprises irradiating the film with an infrared laser.

9. The neural stimulation device of claim 6, wherein the fluorinated polyimide has one of the following repeating units: 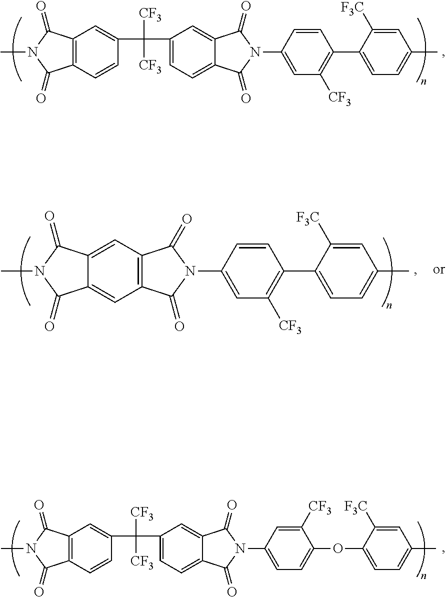
where each instance of n is independently an integer that is at least two.

10. The neural stimulation device of claim 6, wherein the fluorinated polyimide is prepared by thermal imidization of a precursor polyamic acid film.

11. The neural stimulation device of claim 1, wherein the transparent polymer film has an average thickness of 20-300 μm.

12. A method for stimulating a nerve of a subject, comprising placing a neural stimulation device within sufficient proximity to the nerve to thereby stimulate the nerve; wherein the neural stimulation device comprises:
a) a substrate;
b) a transparent polymer film deposited on the substrate; and
c) at least one layer of porous graphene on or within the transparent polymer film;
wherein the at least one layer of porous graphene has the following pore structure:
v) macropores having an average pore size exceeding 50 nm;
vi) mesopores having an average pore size of 2-50 nm;
vii) micropores having an average pore size of 2 nm or less; and
viii) nanopores having an average pore size of less than 100 nm;
wherein the nanopores have a BET specific surface area of at least 300 m²/g.

13. The method of claim 12, wherein the neural stimulation device contacts the nerve.

14. A method for making a neural stimulation device, comprising:
a) depositing a transparent polymer film onto a substrate, wherein the transparent polymer film comprises a fluorinated polyimide having at least one aromatic ring; and
b) graphitizing the fluorinated polyimide to form at least one layer of porous graphene on or within the transparent polymer film.

15. The method of claim 14, wherein the at least one layer of porous graphene has the following pore structure:
a) macropores having an average pore size exceeding 50 nm;
b) mesopores having an average pore size of 2-50 nm;
c) micropores having an average pore size of 2 nm or less; and
d) nanopores having an average pore size of less than 100 nm;
wherein the nanopores have a BET specific surface area of at least 300 m²/g.

16. The method of claim 14, wherein the fluorinated polyimide has one of the following repeating units: 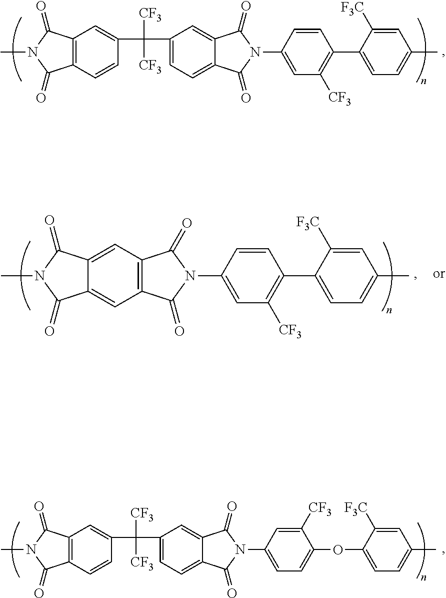
where each instance of n is independently an integer that is at least two.

17. The method of claim 14, wherein the fluorinated polyimide is prepared by thermal imidization of a precursor polyamic acid film.

18. The method of claim 14, wherein the transparent polymer film has an average thickness of 20-300 μm.

19. The method of claim 14, wherein the at least one layer of porous graphene has an average thickness of 10-180 μm.

20. The method of claim 12, wherein graphitizing comprising irradiating the fluorinated polyimide with a CO₂ infrared laser having a wavelength (λ) of 10.6 μm.
